(19) United States
(12) Patent Application Publication
(10) Pub. No.: US 2026/0137725 A1
(43) Pub. Date: May 21, 2026

(54) ANIMAL-DERIVED POLYMER DOT, PREPARATION METHOD AND USE THEREOF

(71) Applicant: China Medical University, Taichung City (TW)

(72) Inventors: Tzong-Yuan Juang, Taichung City (TW); Chang-Cheng Chang, Taichung City (TW); Wen-Ping Jiang, Taipei City (TW); Shu-Ting Tung, New Taipei City (TW)

(73) Assignee: China Medical University, Taichung City (TW)

(21) Appl. No.: 19/396,374

(22) Filed: Nov. 21, 2025

(30) Foreign Application Priority Data
Nov. 21, 2024 (TW) 113144788

(51) Int. Cl. A61K 35/32; A61P 39/06; B82Y 5/00; B82Y 40/00; C01B 21/06

(52) U.S. Cl. CPC A61K 35/32; A61P 39/06; C01B 21/06; B82Y 5/00; B82Y 40/00; C01P 2002/72; C01P 2002/78; C01P 2002/82; C01P 2002/84; C01P 2004/04; C01P 2004/64; C01P 2006/40; C01P 2006/60

(57) ABSTRACT

An animal-derived polymer dot is provided, which comprises a carbon dot represented by Formula (1): CₓO_{y}N_{z} (1), wherein C is carbon, O is oxygen, and N is nitrogen; x, y, and z represent atomic percentages and satisfy the following condition: 55≤x≤75, 15≤y≤35, 10≤z≤30, and x+y+z=100. The carbon dot is obtained from an animal-derived material. A method for preparing the animal-derived polymer dot comprises: adding an animal-derived material into water to form an aqueous solution; and heating the aqueous solution to obtain the animal-derived polymer dot. Also provided is a use of the animal-derived polymer dot in the manufacture of a composition for at least one of anti-inflammatory and anti-oxidant purposes.

### BACKGROUND OF THE INVENTION

### 1. Field of the Invention

The present disclosure relates to animal-derived polymer dots, and more particularly to polymer dots comprising carbon dots derived from an animal-derived material, as well as methods of preparing the polymer dots and uses thereof.

### 2. Description of the Prior Art

Various approaches have been proposed for treating diseases associated with oxidative stress. However, the availability of antioxidant drugs capable of reducing oxidative stress and preventing related diseases remains limited.

Natural enzymes derived from natural substances are known antioxidant materials. Nevertheless, such natural enzymes suffer from disadvantages including high production cost, low yield, structural instability, complex composition, and large molecular size that limits cellular permeability, thereby severely restricting their clinical application.

Accordingly, there remains a strong need in the art for materials and preparation methods capable of overcoming the foregoing problems.

### SUMMARY OF THE INVENTION

To address the foregoing problems, the present disclosure provides an animal-derived polymer dot comprising a carbon dot represented by Formula (1): CₓO_{y}N_{z} (1), where C is carbon, O is oxygen, and N is nitrogen, and x, y, and z represent atomic percentages and satisfy the condition: 55≤x≤75, 15≤y≤35, 10≤z≤30, and x+y+z=100, and wherein the carbon dot is derived from an animal-derived material.

The present disclosure further provides a method of preparing the animal-derived polymer dot, the method comprising: adding an animal-derived material into water to form an aqueous solution; and heating the aqueous solution to obtain the animal-derived polymer dot.

The present disclosure further provides the use of animal-derived polymer dots in the manufacture of a composition for at least one of anti-inflammatory and antioxidant purposes.

In at least one embodiment of the present disclosure, nanoscale animal-derived polymer dots may be produced by a simple, low-cost, and easy preparation method. The resulting animal-derived polymer dots exhibit photostability, low cytotoxicity, biocompatibility, nitric oxide scavenging capability, and excellent antioxidant activity, and may be used in preparing anti-inflammatory and/or antioxidant compositions.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a fluorescence photograph of at least one exemplary embodiment of the present disclosure, illustrating the fluorescence performance of the animal-derived polymer dots.

FIG. 1B is a UV/visible spectrum of at least one exemplary embodiment of the present disclosure, illustrating the average absorbance of the animal-derived polymer dots.

FIG. 2A is a transmission electron microscopy image of at least one exemplary embodiment of the present disclosure, illustrating the morphology and particle size of the animal-derived polymer dots.

FIG. 2B is a schematic diagram illustrating the average particle size distribution of the animal-derived polymer dots of at least one exemplary embodiment of the present disclosure.

FIG. 3A is a schematic diagram illustrating the major elemental components and their contents of the animal-derived polymer dots of at least one exemplary embodiment of the present disclosure.

FIG. 3B is a Fourier transform infrared spectrum of at least one exemplary embodiment of the present disclosure, illustrating the functional groups of the animal-derived polymer dots.

FIG. 4 is a schematic diagram illustrating the diffraction peaks of the animal-derived polymer dots of at least one exemplary embodiment of the present disclosure.

FIG. 5A is a schematic diagram illustrating the effect of the animal-derived polymer dots on the viability of human hepatocellular carcinoma HepG2 cells, according to at least one exemplary embodiment of the present disclosure.

FIG. 5B is a schematic diagram illustrating the effect of the animal-derived polymer dots on the viability of mouse macrophage RAW 264.7 cells, according to at least one exemplary embodiment of the present disclosure.

FIG. 5C is a schematic diagram illustrating the effect of the animal-derived polymer dots on the viability of RAW 264.7 cells induced by lipopolysaccharide (LPS)-induced inflammation, according to at least one exemplary embodiment of the present disclosure. LPS: lipopolysaccharide; PD: polymer dot.

FIG. 6A is a schematic diagram illustrating the nitric oxide scavenging capability of the animal-derived polymer dots in RAW 264.7 cells, according to at least one exemplary embodiment of the present disclosure.

FIG. 6B is a schematic diagram illustrating the nitric oxide scavenging capability of the animal-derived polymer dots in RAW 264.7 cells induced by LPS-induced inflammation, according to at least one exemplary embodiment of the present disclosure. LPS: lipopolysaccharide; PD: polymer dot. **P<0.01 (compared with LPS); ###P<0.001 (compared with control group).

FIG. 7A is a schematic diagram illustrating the capability of the animal-derived polymer dots to scavenge 1-diphenyl-2-picrylhydrazyl (DPPH), according to at least one exemplary embodiment of the present disclosure. **P<0.01 (compared with Preparation Example); ***P<0.001 (compared with Preparation Example).

FIG. 7B is a schematic diagram illustrating the capability of the animal-derived polymer dots to scavenge 2,2′-azino-bis(3-ethylbenzothiazoline-6-sulfonic acid) diammonium salt (ABTS), according to at least one exemplary embodiment of the present disclosure. ***P<0.001 (compared with Preparation Example).

### DETAILED DESCRIPTION

The embodiments of the present disclosure are described below with reference to specific exemplary embodiments. A person having ordinary skill in the art to which the present disclosure pertains may readily understand the spirit, advantages, and effects of the present disclosure based on the contents provided herein. However, the specific embodiments described herein are not intended to limit the present disclosure, and the present disclosure may also be practiced or applied in various other embodiments. The details provided herein may likewise be modified or altered from different perspectives or for different applications without departing from the spirit of the present disclosure.

The proportions, structures, sizes, and other features shown in the drawings attached hereto are provided solely to facilitate understanding of the present disclosure by a person having ordinary skill in the art, and are not intended to limit the scope of practicable embodiments. Accordingly, any changes in proportional relationships, modifications of structures, or adjustments of sizes that do not affect the objectives or performance of the present disclosure are intended to fall within the scope of the technical content disclosed herein.

As used herein, the terms “include,” “comprise,” “contain,” “have,” or variations thereof refer to the presence of the stated features, elements, components, steps, or connections, but do not preclude the presence or addition of one or more other features, elements, components, steps, or connections unless otherwise specified.

Unless expressly stated otherwise herein, the singular forms “a,” “an,” and “the” include plural forms as well. The terms “or” and “/” are used interchangeably with “and/or.”

As used herein, the term “about” refers to variations falling within typical tolerances in the relevant technical field. For example, “about” may be understood as within approximately two standard deviations of a mean value. When “about” precedes a series of numerical values or a numerical range, “about” is understood to modify each individual value in the series or range. The numerical values are intended to encompass variations of 25%, ±20%, ±10%, ±5%, ±1%, ±0.5%, ±0.2%, or ±0.1%. Such variations may occur due to, for example, experimental error; typical deviations in measuring, processing, or handling compounds, compositions, concentrates, or formulations; variations in the source, preparation, or purity of starting materials or components used in the present disclosure; or similar considerations.

The numerical ranges described herein are inclusive and combinable. Any value falling within a stated range may serve as a minimum or maximum value to form sub-ranges. For example, the range “−30 mV to 0 mV” is understood to include any sub-range between −30 mV and 0 mV, such as −29 mV to −0.1 mV, −28 mV to −0.2 mV, and −27 mV to −0.3 mV. In addition, multiple numerical endpoints described herein may be selected as minimum or maximum values to form additional numerical ranges. For example, −20 mV, −10 mV, and −1 mV may be used to derive the ranges −20 mV to −10 mV, −10 mV to −1 mV, or −20 mV to −1 mV.

As used herein, the term “polymer dot” refers to a polymer formed through chemical crosslinking of molecules under thermal treatment, wherein the resulting particle size may be at the nanoscale when dispersed in an aqueous environment. Unless expressly indicated otherwise, the terms “carbon dot” and “carbon nanodot (CND)” are used interchangeably herein. Carbon nanodots have been widely studied in materials science and biomedical applications due to their tunable photoluminescence properties, multicolor emission, photostability, low cost, biocompatibility, low cytotoxicity, high water solubility, and simple synthesis. In addition, materials extracted from natural resources are environmentally friendly, biodegradable, biocompatible, accessible, and low-cost. Accordingly, in at least one embodiment of the present disclosure, animal-derived carbon nanodots are employed.

As used herein, the term “oxidative stress” refers to a condition in which excessive free radicals and reactive oxygen species (ROS) generated during metabolic processes disrupt the balance with the body's antioxidant systems. In some embodiments, the animal-derived polymer dot of the present disclosure exhibits excellent free-radical scavenging capability and may serve as an effective and novel antioxidant that contributes to delaying aging and extending lifespan.

As used herein, the term “nanozyme” refers to nanoparticles having enzyme-like functional properties that serve as substitutes for natural enzymes. Nanozymes may be used for treating diseases such as sepsis, Alzheimer's disease, oral ulcers, cardiovascular diseases, COVID-19, inflammation, or tissue injury, although the present disclosure is not limited thereto. Nanozymes are a novel class of enzyme-functional nanomaterials and exhibit higher stability and more easily regulated catalytic activity compared with natural enzymes. In some embodiments, the animal-derived polymer dots of the present disclosure exhibit biological activity due to their nitrogen-containing structures, particularly antioxidant activity similar to that of natural enzymes (enzyme-like activity). Accordingly, the polymer dots may function as nanozymes and serve as materials for delaying aging and treating diseases caused by oxidative stress.

As used herein, the term “antler” refers to the ossified antlers of animals of the family Cervidae, including sika deer (Cervus nippon Temminck) or red deer (Cervus elaphus Linnaeus), or the antler base shed in the spring of the year following velvet harvest. According to historical literature, antlers possess multiple functions. For example, the Compendium of Materia Medica Volume 2, Deer” records: “When used raw, antlers dissipate heat and promote blood circulation, reduce swelling, and dispel pathogens; when processed, they tonify the kidneys, replenish deficiencies, strengthen vitality, and invigorate blood; when refined into paste, they function primarily as a tonic.” Recent studies have also reported that antlers are rich in proteins, amino acids, carbohydrates, and lipids, and may exhibit therapeutic effects on breast cancer, prostate cancer, acute kidney injury, sexual dysfunction, neuronal injury, cardiac fibrosis, ischemic heart disease, vascular diseases, osteoporosis, and hepatocellular injury.

As used herein, the term “antler gelatin (AG)” refers to a gelatinous substance obtained by decocting and concentrating antlers. A known method for preparing antler gelatin includes slicing the antlers, soaking the slices in water until the water is clear, and repeatedly boiling them in water to obtain a gelatinous solution. A small amount of potassium aluminum sulfate powder is then used to filter and combine the gelatinous solutions. Soybean oil, crystal sugar, and rice wine are added, and the mixture is simmered until a thick paste is formed. After natural solidification, the product is cut into small pieces and air-dried to obtain semi-transparent yellow-brown or red-brown antler gelatin containing a high level of collagen.

As used herein, the term “cluster” refers to an aggregate formed by crosslinking of polymer dots, wherein the aggregated structure is compact and exhibits spectral characteristics different from those of other polymer dots, and possesses unique optical properties.

As used herein, the term “crosslink-enhanced emission (CEE) effect” refers to a luminescence mechanism of polymer dot clusters, in which the fluorescence characteristics are induced or amplified through polymeric chemical crosslinking.

As used herein, the term “d₍₀₀₂₎” refers to the interplanar spacing of a crystal plane of the animal-derived polymer dot corresponding to the graphite (002) plane.

As used herein, the term “d₍₁₀₀₎” refers to the interplanar spacing of a crystal plane of the animal-derived polymer dot corresponding to the graphite (100) plane.

As used herein, the term “at %” refers to atomic percentage (atomic %).

As used herein, the term “subject” refers to an individual, a cell, or a microorganism. In at least one embodiment of the present disclosure, the cell may be a macrophage or a cancer cell, although the present disclosure is not limited thereto. In at least one embodiment of the present disclosure, the microorganism may be Escherichia coli, although the present disclosure is not limited thereto.

As used herein, the term “individual” refers to any vertebrate animal, including but not limited to a human, monkey, mouse, rat, marmot, ferret, rabbit, hamster, cow, horse, pig, deer, dog, cat, fox, wolf, chicken, emu, or ostrich. In at least one embodiment of the present disclosure, the individual is a mammal, including but not limited to a primate (e.g., a human, chimpanzee, gorilla, orangutan, ape, or monkey) or a rodent (e.g., a mouse, rat, marmot, or hamster).

As used herein, the term “effective amount” refers to an amount of an active ingredient (e.g., the animal-derived polymer dot) or a composition containing the active ingredient that is sufficient to produce an effect in a subject in need thereof, such as an antioxidant effect or an anti-inflammatory effect. In some embodiments, the effective amount prevents or reduces the appearance and/or symptoms associated with an undesired condition. The effective amount may vary according to factors known to a person having ordinary skill in the art, including the use of excipients, the route of administration, possible co-administration with other therapeutic treatments, or the condition to be treated, although the present disclosure is not limited thereto.

As used herein, the term “administering” refers to introducing an active component (e.g., animal-derived polymer dots) or a composition containing the active component into a subject by a method or route such that at least a portion of the active ingredient is delivered to a desired site to produce a desired effect. In some embodiments, the active ingredient of the present disclosure may be introduced locally or systemically into the subject by oral administration, parenteral administration, injection administration, subcutaneous administration, sublingual administration, or topical application, although the present disclosure is not limited thereto.

As used herein, the term “pharmaceutically or cosmetically acceptable carrier” refers to a material, medium, or composition that is acceptable for pharmaceutical or cosmetic use. Examples include, but are not limited to, solid or liquid fillers, binders, diluents, preservatives, biocompatible solvents, disintegrants, lubricants, suspending agents, flavoring agents, capsule materials, thickeners, acids, surfactants, chelating agents, wetting agents, or any combination thereof. In some embodiments, each component is “pharmaceutically or cosmetically acceptable,” meaning that it is compatible with other ingredients of cosmetic or pharmaceutical formulations and is suitable for contact with organs or tissues of a subject (e.g., a mammal) without causing significant toxicity, allergic reaction, irritation, immunogenicity, or other complications or problems. See, for example: Remington: The Science and Practice of Pharmacy, 22nd ed., Allen, Ed., Philadelphia, PA, 2012; Handbook of Pharmaceutical Excipients, 7th ed., Rowe et al., Eds., The Pharmaceutical Press and the American Pharmaceutical Association, 2012; Handbook of Pharmaceutical Additives, 3rd ed., Ash and Ash, Eds., Gower Publishing Company, 2007; Pharmaceutical Preformulation and Formulation, 2nd ed., Gibson, Ed., CRC Press LLC, Boca Raton, FL, 2009.

In at least one embodiment of the present disclosure, C is carbon, O is oxygen, and N is nitrogen, and x, y, and z represent atomic percentages that satisfy the following condition: 55≤x≤70, 20≤y≤35, 10≤z≤25, and x+y+z=100.

In at least one embodiment of the present disclosure, based on the total atomic percentage of the carbon dot, the carbon dot may contain about 55 at % to about 75 at % of carbon, for example, about 55.5 at % to about 74.5 at %, about 56 at % to about 74 at %, or about 56.5 at % to about 73.5 at %. In some embodiments, the atomic percentage of carbon may be selected from about 55 at %, 55.5 at %, 56 at %, 56.5 at %, 57 at %, 57.5 at %, 58 at %, 58.5 at %, 59 at %, 59.5 at %, 60 at %, 60.5 at %, 61 at %, 61.5 at %, 62 at %, 62.5 at %, 63 at %, 63.5 at %, 64 at %, 64.5 at %, 65 at %, 65.5 at %, 66 at %, 66.5 at %, 67 at %, 67.5 at %, 68 at %, 68.5 at %, 69 at %, 69.5 at %, 70 at %, 70.5 at %, 71 at %, 71.5 at %, 72 at %, 72.5 at %, 73 at %, 73.5 at %, 74 at %, 74.5 at %, or 75 at %. Although the present disclosure is not limited thereto.

In at least one embodiment of the present disclosure, based on the total atomic percentage of the carbon dot, the carbon dot may contain about 15 at % to about 35 at % of oxygen, for example, about 15.5 at % to about 34.5 at %, about 16 at % to about 34 at %, or about 16.5 at % to about 33.5 at %. In some embodiments, the atomic percentage of oxygen may be selected from about 15 at %, 15.5 at %, 16 at %, 16.5 at %, 17 at %, 17.5 at %, 18 at %, 18.5 at %, 19 at %, 19.5 at %, 20 at %, 20.5 at %, 21 at %, 21.5 at %, 22 at %, 22.5 at %, 23 at %, 23.5 at %, 24 at %, 24.5 at %, 25 at %, 25.5 at %, 26 at %, 26.5 at %, 27 at %, 27.5 at %, 28 at %, 28.5 at %, 29 at %, 29.5 at %, 30 at %, 30.5 at %, 31 at %, 31.5 at %, 32 at %, 32.5 at %, 33 at %, 33.5 at %, 34 at %, 34.5 at %, or 35 at %. Although the present disclosure is not limited thereto.

In at least one embodiment of the present disclosure, based on the total atomic percentage of the carbon dots, the carbon dots may contain about 10 at % to about 30 at % of nitrogen, for example, about 10.5 at % to about 29.5 at %, about 11 at % to about 29 at %, or about 11.5 at % to about 28.5 at %. In some embodiments, the atomic percentage of nitrogen may be selected from about 10 at %, 10.5 at %, 11 at %, 11.5 at %, 12 at %, 12.5 at %, 13 at %, 13.5 at %, 14 at %, 14.5 at %, 15 at %, 15.5 at %, 16 at %, 16.5 at %, 17 at %, 17.5 at %, 18 at %, 18.5 at %, 19 at %, 19.5 at %, 20 at %, 20.5 at %, 21 at %, 21.5 at %, 22 at %, 22.5 at %, 23 at %, 23.5 at %, 24 at %, 24.5 at %, 25 at %, 25.5 at %, 26 at %, 26.5 at %, 27 at %, 27.5 at %, 28 at %, 28.5 at %, 29 at %, 29.5 at %, or 30 at %. Although the present disclosure is not limited thereto.

In at least one embodiment of the present disclosure, the carbon dots may have a zeta potential ranging from about −30 mV to about 0 mV, for example, from about −25 mV to about −0.1 mV, from about −20 mV to about −0.2 mV, or from about −15 mV to about −0.3 mV.

In some embodiments, the zeta potential may be selected from about −30 mV, −25 mV, −20 mV, −15 mV, −10 mV, −5 mV, −2 mV, −1.9 mV, −1.8 mV, −1.7 mV, −1.6 mV, −1.5 mV, −1.4 mV, −1.3 mV, −1.2 mV, −1.1 mV, −1.0 mV, −0.9 mV, −0.8 mV, −0.7 mV, −0.6 mV, −0.5 mV, −0.4 mV, −0.3 mV, −0.2 mV, −0.1 mV, or 0 mV. However, the present disclosure is not limited thereto.

In at least one embodiment of the present disclosure, the particle size of the carbon dots may range from about 1 nm to about 90 nm. Examples include, but are not limited to, from about 5 nm to about 85 nm, from about 10 nm to about 80 nm, or from about 15 nm to about 75 nm. In some embodiments, the particle size may be about 1 nm, 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, or 90 nm, although the present disclosure is not limited thereto.

In at least one embodiment of the present disclosure, the X-ray powder diffraction pattern of the carbon dots may include characteristic peaks at 20 values of 19.32°, 19.96°, 20.68°, 21.72°, 21.76°, 25.16°, 25.20°, 29.12°, 29.20°, 35.88°, 35.96°, 38.92°, and/or 39.04°±0.2°.

In at least one embodiment of the present disclosure, the interplanar spacing of the carbon dots corresponding to the (002) plane of graphite may range from about 3 Å to about 5 Å. Examples include, but are not limited to, from about 3.1 Å to about 4.9 Å, from about 3.2 Å to about 4.8 Å, or from about 3.3 Å to about 4.7 Å. In some embodiments, the interplanar spacing corresponding to the (002) plane of graphite may be about 3.1 Å, 3.2 Å, 3.3 Å, 3.4 Å, 3.5 Å, 3.6 Å, 3.7 Å, 3.8 Å, 3.9 Å, 4.0 Å, 4.1 Å, 4.2 Å, 4.3 Å, 4.4 Å, 4.5 Å, 4.6 Å, 4.7 Å, 4.8 Å, 4.9 Å, or 5.0 Å, although the present disclosure is not limited thereto.

In at least one embodiment of the present disclosure, the interplanar spacing of the carbon dots corresponding to the (100) plane of graphite may range from about 1 Å to about 3 Å. Examples include, but are not limited to, from about 1.1 Å to about 2.9 Å, from about 1.2 Å to about 2.8 Å, or from about 1.3 Å to about 2.7 Å. In some embodiments, the interplanar spacing corresponding to the (100) plane of graphite may be about 1.1 Å, 1.2 Å, 1.3 Å, 1.4 Å, 1.5 Å, 1.6 Å, 1.7 Å, 1.8 Å, 1.9 Å, 2.0 Å, 2.1 Å, 2.2 Å, 2.3 Å, 2.4 Å, 2.5 Å, 2.6 Å, 2.7 Å, 2.8 Å, 2.9 Å, or 3.0 Å, although the present disclosure is not limited thereto.

In at least one embodiment of the present disclosure, the carbon dots are formed by heating the animal-derived material using a hydrothermal method.

In at least one embodiment of the present disclosure, the animal may be a deer.

In at least one embodiment of the present disclosure, the animal-derived material may be deerhorn glue.

In at least one embodiment of the present disclosure, after heating the aqueous solution, the method may further include treating the aqueous solution by a hydrothermal method.

In at least one embodiment of the present disclosure, treating the aqueous solution by a hydrothermal method may include heating the aqueous solution to a temperature of 150° C. to 250° C. under a pressure of 100 psi to 120 psi, and maintaining the heating temperature for a heating time of 1 hour to 10 hours.

In at least one embodiment of the present disclosure, the pressure may be from about 100 psi to about 120 psi, for example, but not limited to, about 101 psi to about 117 psi, about 102 psi to about 118 psi, or about 103 psi to about 119 psi. In some embodiments, the pressure may be about 100 psi, 101 psi, 102 psi, 103 psi, 104 psi, 105 psi, 106 psi, 107 psi, 108 psi, 109 psi, 110 psi, 111 psi, 112 psi, 113 psi, 114 psi, 115 psi, 116 psi, 117 psi, 118 psi, 119 psi, or 120 psi, although the present disclosure is not limited thereto.

In at least one embodiment of the present disclosure, the heating temperature may be from about 150° C. to about 250° C., for example, but not limited to, about 155° C. to about 245° C., about 160° C. to about 240° C., or about 165° C. to about 235° C. In some embodiments, the heating temperature may be about 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., or 250° C., although the present disclosure is not limited thereto.

In at least one embodiment of the present disclosure, the heating time may be from about 1 hour to about 10 hours, for example, but not limited to, about 1.5 to about 9.5 hours, about 2 to about 9 hours, or about 2.5 to about 8.5 hours. In some embodiments, the heating time may be about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, or 10 hours, although the present disclosure is not limited thereto.

In at least one embodiment of the present disclosure, the synthesis yield of the animal-derived polymer dots may be about 5% to about 95%, about 20% to about 90%, about 30% to about 80%, or about 40% to about 70%, although the present disclosure is not limited thereto. In some embodiments, the synthesis yield of the animal-derived polymer dots may be about 5%, 10%, 15%, 20%, 25%, 30%, 35%, 40%, 45%, 50%, 55%, 60%, 65%, 70%, 75%, 80%, 85%, 90%, or 95%.

In at least one embodiment of the present disclosure, the recovery rate of the animal-derived polymer dots may be about 70% to about 99%, about 75% to about 98%, about 80% to about 97%, or about 85% to about 96%, although the present disclosure is not limited thereto. In some embodiments, the recovery rate of the animal-derived polymer dots may be about 70%, 75%, 80%, 85%, 86%, 87%, 88%, 89%, 90%, 91%, 92%, 93%, 94%, 95%, 96%, 97%, 98%, or 99%.

In at least one embodiment of the present disclosure, a method for inhibiting inflammation and/or inhibiting oxidation is provided, the method comprises administering to a subject in need thereof an effective amount of the animal-derived polymer dots.

In some embodiments, the present disclosure may utilize heating and/or hydrothermal treatment of the animal-derived material to obtain gram-scale quantities of the animal-derived polymer dots.

The animal-derived polymer dots of the present disclosure exhibit favorable fluorescence properties and antioxidant activity, enabling their use as diagnostic probes or therapeutic diagnostic agents in bioimaging applications. The animal-derived polymer dots may also be used in treating oxidative stress-related diseases, including, but not limited to, inflammatory skin disorders.

The animal-derived polymer dots of the present disclosure may overcome clinical application limitations associated with conventional antioxidant materials (such as natural enzymes derived from natural sources) including high cost, low yield, unstable structure, complex composition, and large molecular size that restricts cellular permeability.

In addition, due to their antioxidant activity, excellent hydrophilicity, and favorable biocompatibility, the polymer dots of the present disclosure may be readily applied by wiping or spraying.

### EXAMPLES

The following provide exemplary and detailed descriptions of specific embodiments of the present disclosure, which should not be interpreted as limiting the scope of the present disclosure.

### Preparation Example

Five grams of deerhorn glue (purchased from Zhi Xiang Ginseng Medicine Co., Ltd.) were added to 100 mL of double-distilled water, heated, and uniformly stirred until dissolved to form a homogeneous mixed solution, thereby obtaining the animal-derived polymer dots of the preparation example.

### Example 1

The homogeneous mixed solution of the preparation example was placed into a high-temperature and high-pressure reactor, and nitrogen gas was introduced into the reactor. The reactor was then placed in a glycerol bath and heated to 180° C. under a pressure of 110 psi, and the temperature was maintained for 3 hours to allow the heating reaction to proceed. Upon completion of the heating reaction, the solution was centrifuged at 6000 rpm for 30 minutes at room temperature, and the resulting supernatant was collected. The supernatant was filtered through a 0.22 m membrane to remove impurities. Then, the filtered supernatant was concentrated by a vacuum concentrator, and subsequently vacuum-dried at 50° C. to constant weight, thereby obtaining the animal-derived polymer dots of Example 1.

### Example 2

The polymer dots of Example 2 were prepared using the same procedure as described in Example 1, except that the heating time was changed to 6 hours.

### Example 3

The polymer dots of Example 3 were prepared using the same procedure as described in Example 1, except that the heating temperature was changed to 220° C.

### Example 4

The polymer dots of Example 4 were prepared using the same procedure as described in Example 3, except that the heating time was changed to 6 hours.

### Analysis and Results

### 1. Fluorescence Performance

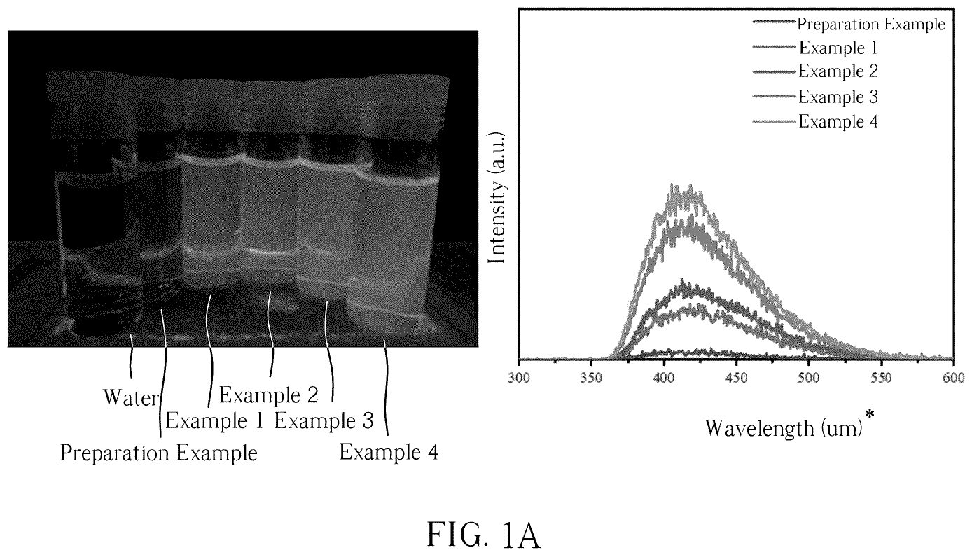

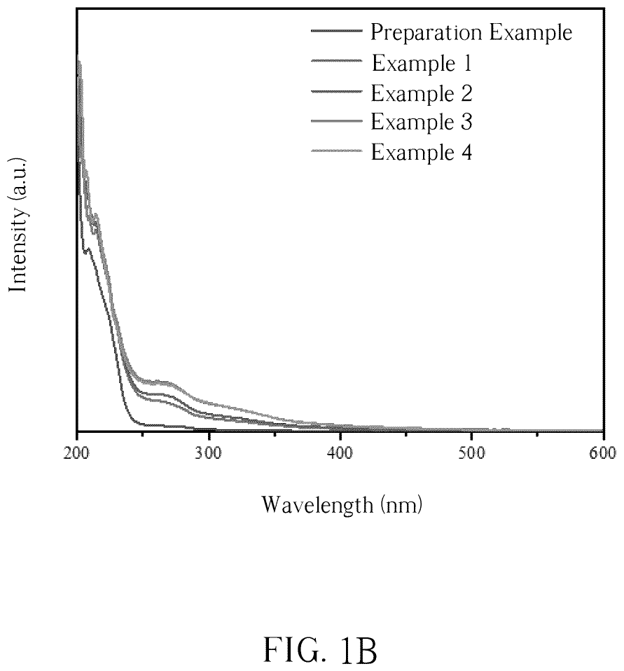

The polymer dots derived from an animal material of the preparation example and of Examples 1 to 4 were added to double-distilled water to obtain solution samples at a concentration of 0.25 mg/mL, respectively. The solutions were irradiated with a UV lamp at a wavelength of 365 nm, and their absorption spectra were measured using a fluorescence spectrometer (LS-50B, PerkinElmer) to evaluate the fluorescence properties of the animal-derived polymer dots. The results are shown in FIG. 1A.

As shown in the left portion of FIG. 1A, the solution samples of Examples 1 to 4 shows significant blue fluorescence. The right portion of FIG. 1A shows the overlaid fluorescence spectra of the preparation example and the samples of Examples 1 to 4. As illustrated, all samples exhibited fluorescence intensity, and the fluorescence intensity increased as the temperature and pressure used during preparation increased. These results confirm that the animal-derived polymer dots of the present disclosure exhibit excellent fluorescence performance.

The average absorbance values of the solution samples were subsequently measured using a UV/visible spectrophotometer (UV-1700, Shimadzu), and the results are shown in FIG. 1B. In addition, the fluorescence quantum yield (QY) was calculated according to Equation (A) below, and the results are summarized in Table 1:

$${Φ}_{A G} = {Φ}_{s t} \times ( {I}_{A G} / {I}_{s t} ) \times {( η / {η}_{s t} )}^{2} \qquad Equation ( A )$$

AG: AG or AG-CNDs sample
   st: quinine sulfate standard diluted in 0.1 M H₂SO₄
   Φ: quantum yield (Φₛₜ=0.54)
I: the slope of the curve generated from the integrated area of the fluorescence emission spectrum versus the UV/vis absorbance
   η: refractive index of the solvent

TABLE 1

| | QY(%) |
|---|---|
| Preparation Example | 4.9 |
| Example 1 | 7.0 |
| Example 2 | 7.7 |
| Example 3 | 7.9 |
| Example 4 | 8.4 |

Referring to FIG. 1B and Table 1, the calculated fluorescence quantum yields of the animal-derived polymer dots demonstrate that both the heating temperature and the heating duration of the hydrothermal process affect the UV/visible absorption intensity and the fluorescence quantum yield. As the heating temperature increases and/or the heating time is extended, the UV/visible absorption intensity correspondingly increases, and the fluorescence quantum yield likewise increases. Accordingly, the present disclosure enables the animal-derived polymer dots to achieve an improved fluorescence quantum yield of about 4% to about 10%.

### 2. Morphology, Particle Size, Water Solubility, and Zeta Potential

### 2.1 Transmission Electron Microscopy (TEM) Analysis

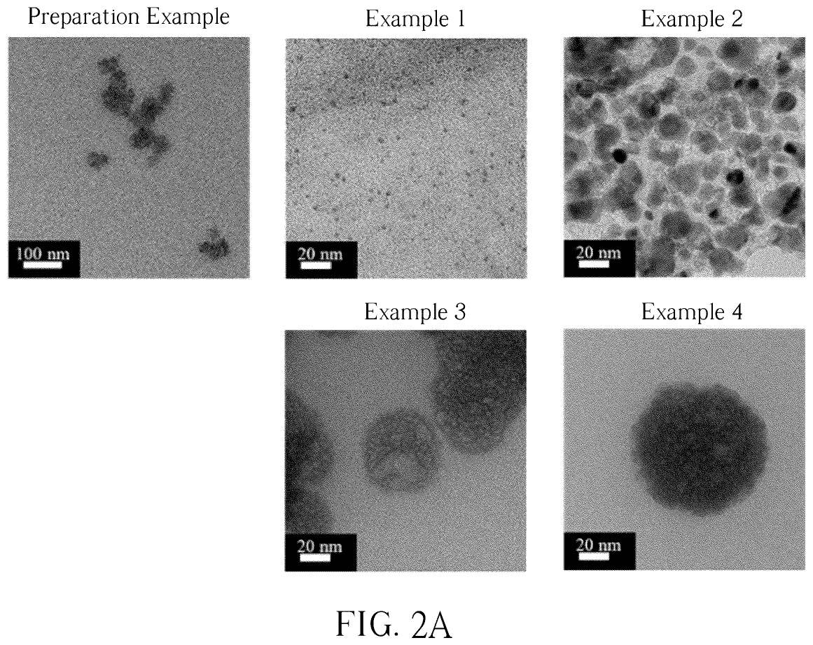

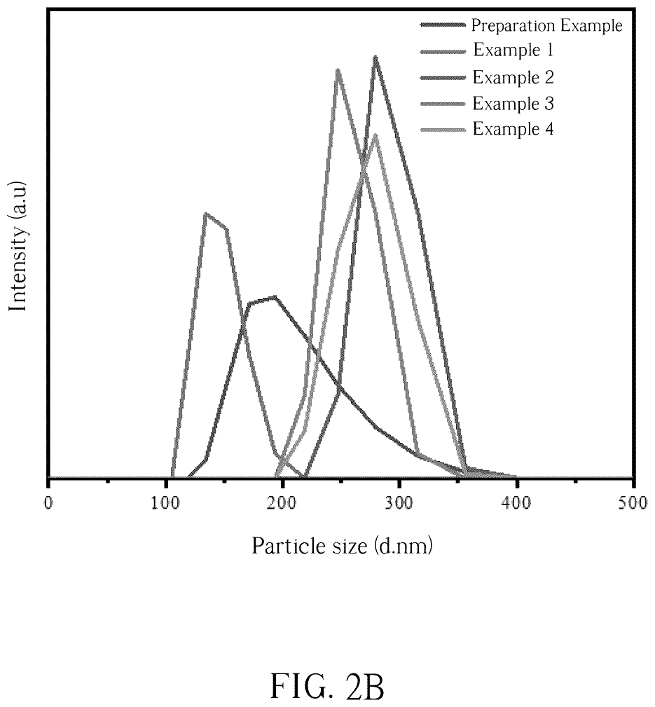

The polymer dots derived from an animal material of the preparation example and Examples 1 to 4 were added to double distilled water to obtain solution samples at a concentration of 1 mg/mL, respectively. A carbon-coated copper grid was immersed in each solution sample for 2 minutes. The grid was then removed using tweezers, gently swung back and forth twice, and excess solution along the grid edge was absorbed. The grid was placed in a desiccator for 12 to 15 hours to air-dry, followed by TEM imaging analysis using a transmission electron microscope (JEM-1400 FLASH, JEOL). The results are shown in FIG. 2A, and the average particle sizes are summarized in Table 2.

Referring to the upper-left portion of FIG. 2A and Table 2, the animal-derived polymer dots of the preparation example exhibited an average particle size of about 100 nm to about 500 nm, displayed granular or sheet-like morphology, and showed good water solubility. Referring to the upper-middle, upper-right, lower-middle, and lower-right portions of FIG. 2A and Table 2, the animal-derived polymer dots of Examples 1 to 4 exhibited average particle sizes of about 2 nm to about 5 nm, about 9 nm to about 20 nm, about 25 nm to about 40 nm, and about 60 nm to about 85 nm, respectively. All exhibited spherical morphology and good water solubility.

### 2.2 Dynamic Light Scattering (DLS) Analysis

The polymer dots derived from an animal material of the preparation example and Examples 1 to 4 were individually added to double-distilled water to obtain solution samples having a concentration of 1 mg/mL. The solution samples were placed into plastic cuvettes and analyzed using a dynamic light scattering (DLS) nanoparticle size analyzer (Zetasizer Nano ZS90, Malvern) to determine the average particle size distribution and zeta potential of the animal-derived polymer dots. The results are shown in FIG. 2B, and the average particle size distribution and zeta potential values are listed in Table 2.

Referring to FIG. 2B and Table 2, the average particle size distributions of the animal-derived polymer dots of the preparation example and Examples 1 to 4 were approximately 190.5±9.4 nm, 137.7±2.7 nm, 273.4±3.8 nm, 240.9±3.3 nm, and 256.7±2.6 nm, respectively. The difference between the average particle size and the average particle size distribution is attributable to the fact that the DLS method measures aggregates of animal-derived polymer dots as a single nanocarbon dot, thereby resulting in a broader particle size distribution.

As further shown in Table 2, the average zeta potentials of the animal-derived polymer dots of the preparation example and Examples 1 to 4 were approximately −0.2±0.0 mV, −1.4±0.0 mV, −26.3±0.4 mV, −11.8±0.7 mV, and −21.5±0.4 mV, respectively. The results indicate that the heating temperature and heating duration of the hydrothermal process both influence the zeta potential. Higher heating temperatures and/or longer heating durations yield larger absolute zeta potential values, indicating that the animal-derived polymer dots can maintain a stable dispersed state for a longer period of time. Accordingly, the present disclosure provides animal-derived polymer dots exhibiting improved dispersion stability.

TABLE 2

| | | Particle Size | |
|---|---|---|---|
| | Particle size | Distribution | Zeta Potential |
| | (nm) | (nm) | (mV) |
| Preparation | 100 to 500 | 190.5 ± 9.4 | −0.2 ± 0.0 |
| Example | | | |
| Example 1 | 2 to 5 | 137.7 ± 2.7 | −1.4 ± 0.0 |
| Example 2 | 9 to 20 | 273.4 ± 3.8 | −26.3 ± 0.4 |
| Example 3 | 25 to 40 | 240.9 ± 3.3 | −11.8 ± 0.7 |
| Example 4 | 65 to 85 | 256.7 ± 2.6 | −21.5 ± 0.4 |

### 3. Major Elemental Components, Their Contents, and Bonding Strength

### 3.1 X-ray Photoelectron Spectroscopy (XPS) Analysis

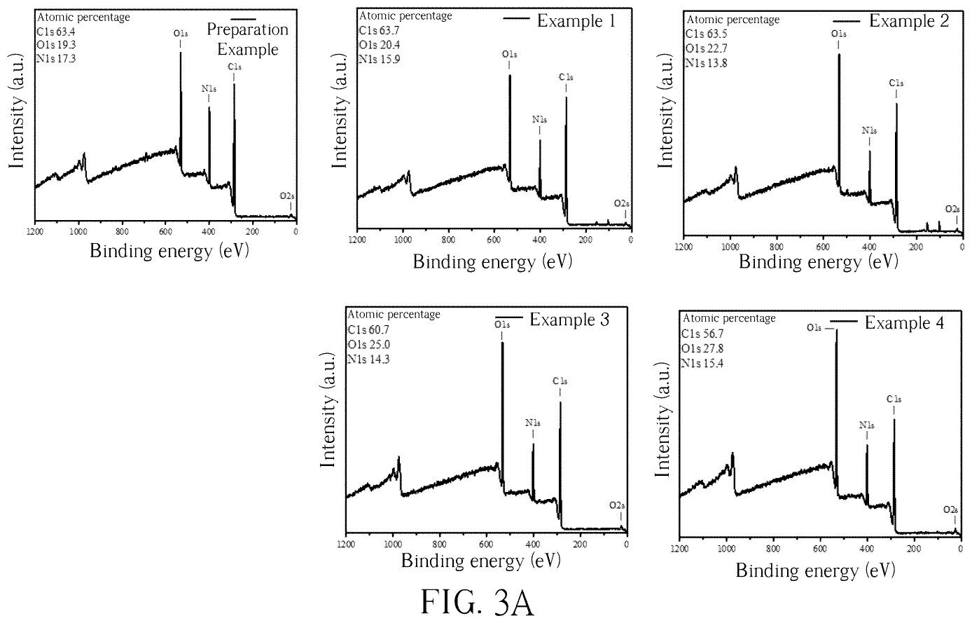

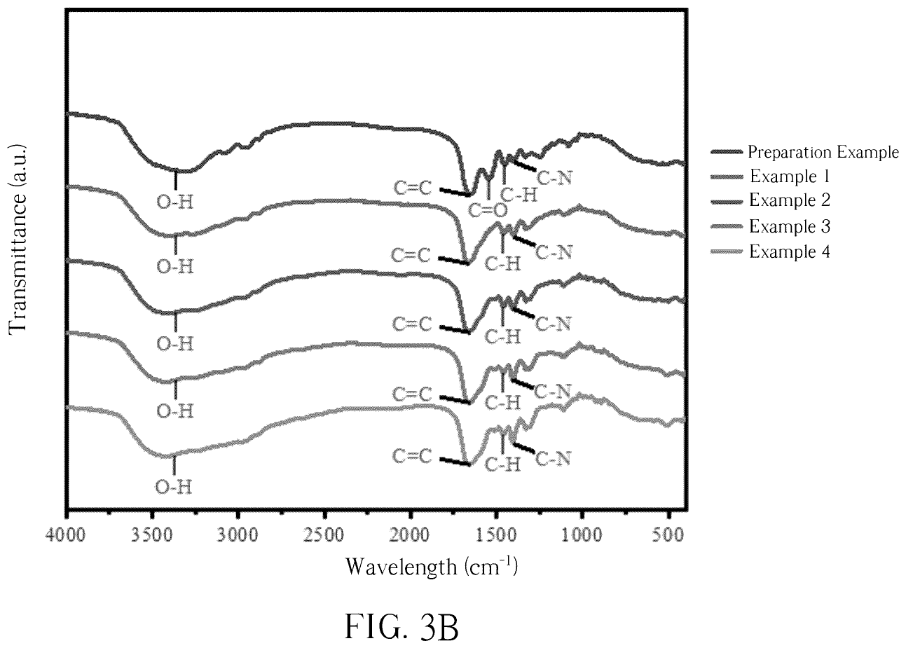

One to two drops of double-distilled water were added to the animal-derived polymer dots of the preparation example and Examples 1 to 4 to obtain viscous samples. Glass slides were cut into pieces measuring 0.5 cm×0.5 cm, and each sample was coated onto a glass slide. The coated slides were placed in a vacuum oven for 24 hours to dry completely. The surface compositions were then analyzed using an X-ray photoelectron spectrometer (PHI 5000 VersaProbe/Scanning ESCAMicroprobe, ULVAC-PHI). The results are shown in FIG. 3A, and the contents of the respective components are listed in Table 3.

TABLE 3

| | Carbon (%) | Oxygen (%) | Nitrogen (%) |
|---|---|---|---|
| Preparation Example | 63.4 | 19.3 | 17.3 |
| Example 1 | 63.7 | 20.4 | 15.9 |
| Example 2 | 63.5 | 22.7 | 13.8 |
| Example 3 | 60.7 | 25.0 | 14.3 |
| Example 4 | 56.8 | 27.8 | 15.4 |

Referring to FIG. 3A, the surfaces of the animal-derived polymer dots of the preparation example and Examples 1 to 4 are all composed of carbon (C), oxygen (O), and nitrogen (N) elements. In addition, as shown in Table 3, increasing the heating temperature and/or prolonging the heating time in the hydrothermal process results in an increase in oxygen content and a corresponding decrease in nitrogen content.

### 3.2 Fourier-Transform Infrared Spectroscopy (FTIR) Analysis

The animal-derived polymer dots of the preparation example and Examples 1 to 4 were added to double distilled water to obtain solution samples at a concentration of 50 mg/mL, respectively. Potassium bromide (KBr) was dried and ground into a fine powder, and then compressed into a light-transmitting pellet using a tablet press. One drop of each solution sample was applied to the KBr pellet and allowed to air-dry. The pellets were then analyzed using a Fourier-transform infrared spectrometer (IRAffinity-1S, Shimadzu) to determine the surface functional groups. The results are shown in FIG. 3B.

As shown in FIG. 3B, the O—H stretching vibration,

C═C stretching vibration, C═O stretching vibration, C—H bending vibration, and C—N stretching vibration of the animal-derived polymer dots of the preparation example occur at 3000-3700 cm⁻¹, 1649 cm⁻¹, 1548 cm⁻¹, 1453 cm⁻¹, and 1401 cm⁻¹, respectively. For the animal-derived polymer dots of Examples 1 to 4, the C═O stretching vibration is attenuated, while the O—H stretching vibration, C═C stretching vibration, C—H bending vibration, and C—N stretching vibration occur at 3000-3700 cm⁻¹, 1658-1667 cm⁻¹, 1459-1461 cm⁻¹, and 1402-1408 cm⁻¹, respectively. These results indicate that the animal-derived polymer dots of the preparation example may include O—H, C═C, C═O, C—H, and C—N functional groups, while those of Examples 1 to 4 may include O—H, C═C, C—H, and C—N functional groups, but do not include the C═O functional group.

### 4. Crystal Structure

### 4.1 X-Ray Diffraction (XRD) Analysis

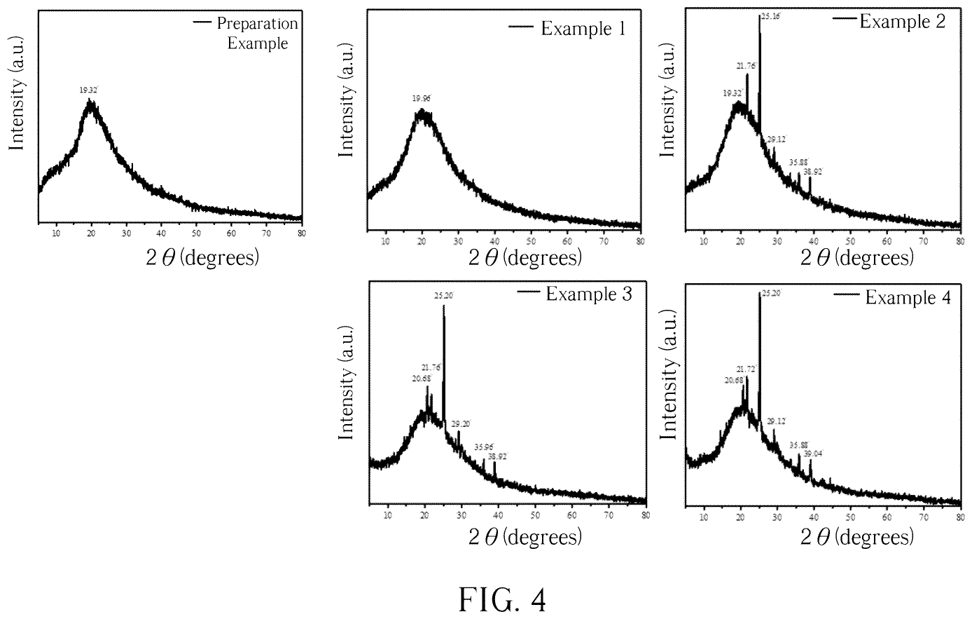

One to two drops of double distilled water were added to the animal-derived polymer dots of the preparation example and Examples 1 to 4 to obtain viscous samples. Microscope slides were cut into 1 cm×1 cm pieces, and each sample was coated onto a slide. The slides were then placed in a vacuum oven for 24 hours until completely dried. The crystal structures were subsequently analyzed using an X-ray diffractometer (D8 DISCOVER, BRUKER), and the results are shown in FIG. 4. Thereafter, in accordance with Bragg's law shown in Equation (B), the interplanar spacings d₍₀₀₂₎ and d₍₁₀₀₎ of the animal-derived polymer dots of the preparation example and Examples 1 to 4 were calculated, and the resulting d₍₀₀₂₎ and d₍₁₀₀₎ values are summarized in Table 4.

$$n λ = 2 d sin ( θ ) \qquad Equation ( B )$$

n: a positive integer
λ: wavelength of the incident X-ray (λ=1.5418 Å)
d: interplanar spacing
θ: diffraction angle

TABLE 4

| | Diffraction | | Diffraction | |
|---|---|---|---|---|
| | Peak (°) | d₍₀₀₂₎(Å) | Peak (°) | d₍₁₀₀₎(Å) |
| Preparation | 19.32 ± 0.2 | 4.6 | — | — |
| Example | | | | |
| Example 1 | 19.96 ± 0.2 | 4.4 | — | — |
| Example 2 | 19.32 ± 0.2 | 4.6 | 29.12 ± 0.2 | 3.1 |
| | 21.76 ± 0.2 | 4.1 | 35.88 ± 0.2 | 2.5 |
| | 25.16 ± 0.2 | 3.5 | 38.92 ± 0.2 | 2.3 |
| Example 3 | 20.68 ± 0.2 | 4.3 | 29.20 ± 0.2 | 3.1 |
| | 21.76 ± 0.2 | 4.1 | 35.96 ± 0.2 | 2.5 |
| | 25.20 ± 0.2 | 3.5 | 38.92 ± 0.2 | 2.3 |
| Example 4 | 20.68 ± 0.2 | 4.3 | 29.12 ± 0.2 | 3.1 |
| | 21.72 ± 0.2 | 4.1 | 35.88 ± 0.2 | 2.5 |
| | 25.20 ± 0.2 | 3.5 | 39.04 ± 0.2 | 2.3 |

Referring to FIG. 4 and Table 4, the animal-derived polymer dots of the Preparation Example and Examples 1 to 4 exhibit characteristic diffraction peaks at 20 values of about 180 to about 260 and about 280 to about 40°, corresponding to the graphite (002) and graphite (100) crystal planes, respectively. The results show that, as the hydrothermal heating temperature increases and/or the heating time is extended, the characteristic peaks undergo slight shifts and the interplanar spacings change accordingly, confirming that the heating and hydrothermal conditions affect the crystal structure of the animal-derived polymer dots.

### 5. Cytotoxicity

The animal-derived polymer dots of the Preparation Example and Examples 1 to 4 were added to double-distilled water to obtain solutions having a concentration of 20 mg/mL, respectively. Each solution was diluted with cell culture medium to prepare samples at concentrations of 0 μg/mL (control), 125 μg/mL, 250 μg/mL, 500 μg/mL, and 1000 μg/mL.

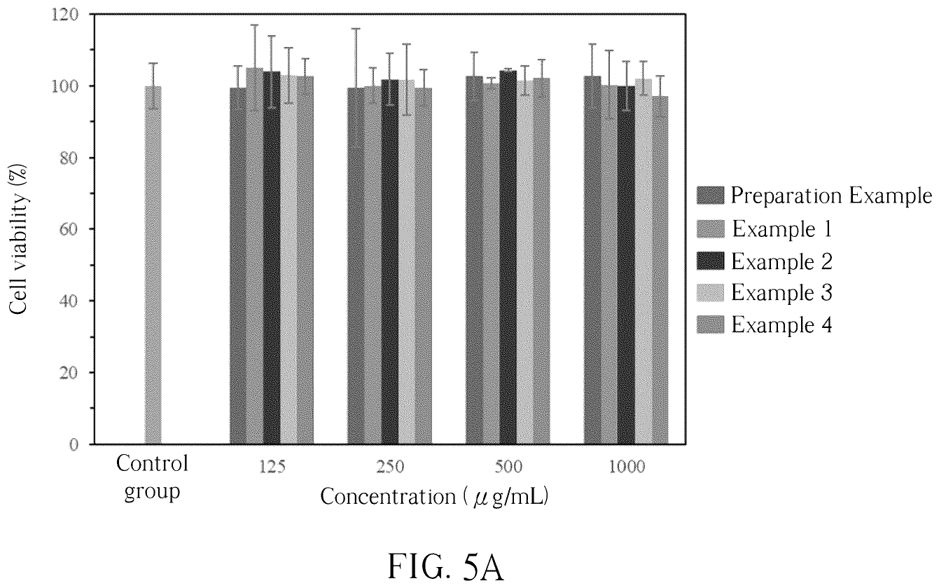

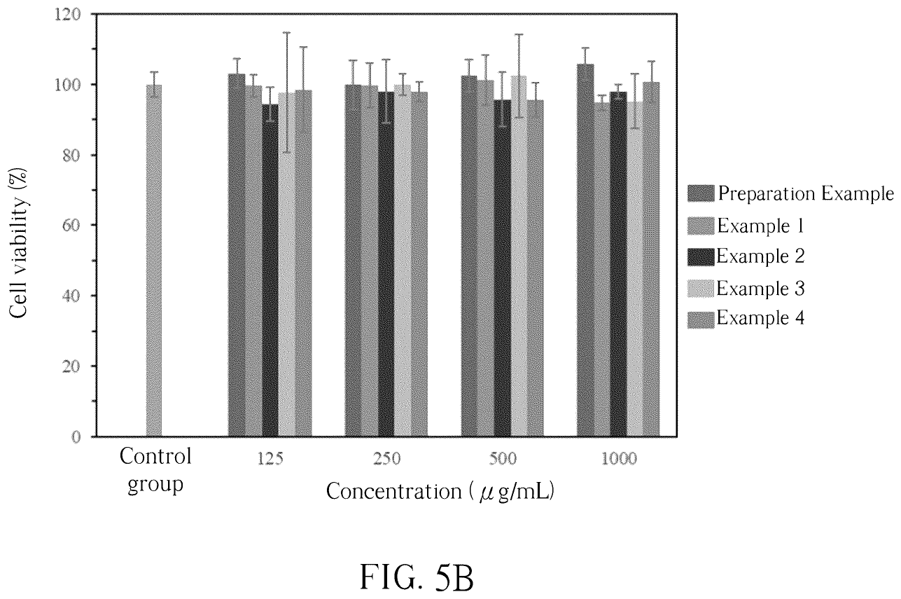

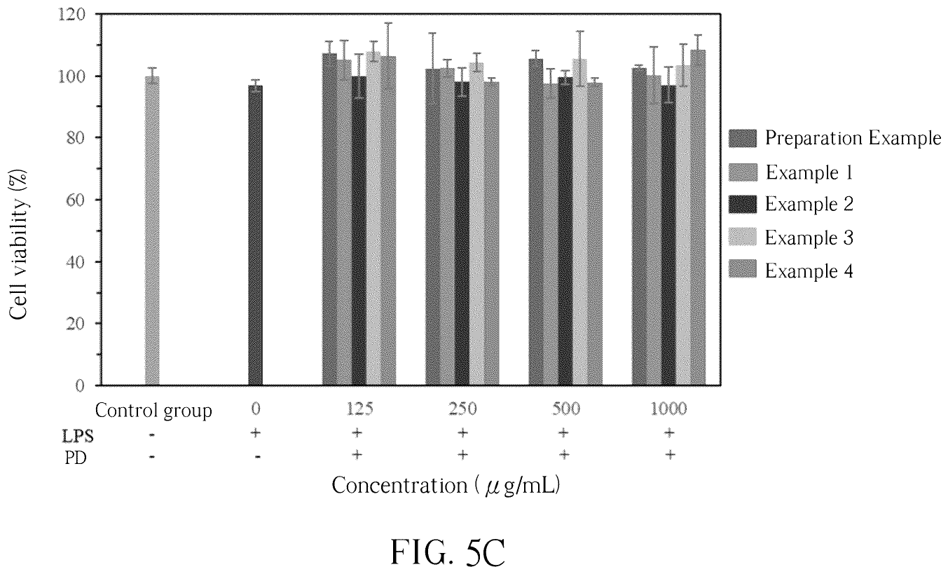

Human hepatocellular carcinoma HepG2 cells, mouse macrophage RAW 264.7 cells, and RAW 264.7 cells stimulated in vitro with lipopolysaccharide (LPS) to induce nitric oxide (NO) production were seeded at 5×10⁴ cells per well in 96-well plates, respectively. The cells were incubated at 37° C. for 24 hours to allow adhesion. After adhesion, double-distilled water or the various sample concentrations were added, followed by incubation at 37° C. for an additional 24 hours. The culture medium was then replaced with medium containing 0.5 mg/mL of 3-(4,5-dimethylthiazol-2-yl)-2,5-diphenyltetrazolium bromide (MTT), and the cells were incubated for 4 hours at 37° C. The supernatant was removed, 200 L of dimethyl sulfoxide (DMSO) was added, and the absorbance at 570 nm (OD570) was measured using a multi-mode microplate spectrophotometer (M2e, Molecular Devices). Cytotoxicity and biocompatibility toward HepG2 cells and RAW 264.7 cells were evaluated, and cell viability was calculated according to Equation (C). The results are shown in FIGS. 5A to 5C:

$$Cell viability ( \% ) = ( {A}_{s} \div {A}_{c} ) \times 100 \% \qquad Equation ( C )$$

Aₛ: the absorbance value of the cells after incubation with the sample of the Preparation Example or any of the Examples.
A_{c}: the absorbance value of the cells after incubation with double-distilled water.

Referring to FIG. 5A, when the concentration of the animal-derived polymer dots of the Preparation Example and Examples 1 to 4 was 1000 μg/mL, the cell viability of HepG2 cells was greater than 80% in all cases. These results indicate that the animal-derived polymer dots of the present disclosure do not induce acute hepatotoxicity and exhibit low cytotoxicity and good biocompatibility toward HepG2 cells.

Referring to FIG. 5B, when the concentration of the animal-derived polymer dots of the Preparation Example and Examples 1 to 4 was 1000 μg/mL, the cell viability of RAW 264.7 cells was greater than 80% in all cases. These results indicate that the animal-derived polymer dots of the present disclosure exhibit low cytotoxicity and good biocompatibility toward RAW 264.7 cells.

Referring to FIG. 5C, when the concentration of the animal-derived polymer dots of the Preparation Example and Examples 1 to 4 was 1000 μg/mL, the cell viability of RAW 264.7 cells induced with LPS to produce NO was greater than 80% in all cases. These results indicate that the animal-derived polymer dots of the present disclosure exhibit low cytotoxicity and good biocompatibility toward RAW 264.7 cells under LPS-induced inflammatory conditions.

### 6. Nitric Oxide Scavenging Capability

The animal-derived polymer dots of the Preparation Example and Examples 1 to 4 were added to double-distilled water to obtain solutions at a concentration of 20 mg/mL, respectively. Each solution was then diluted with cell culture medium to prepare samples having concentrations of 0 μg/mL (control), 125 μg/mL, 250 μg/mL, 500 μg/mL, and 1000 μg/mL. A 100 ng/mL LPS solution served as the positive control.

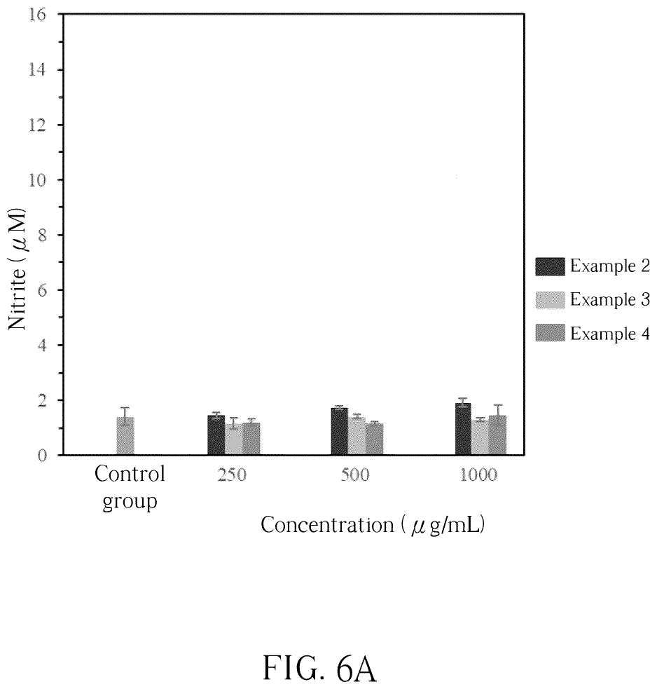

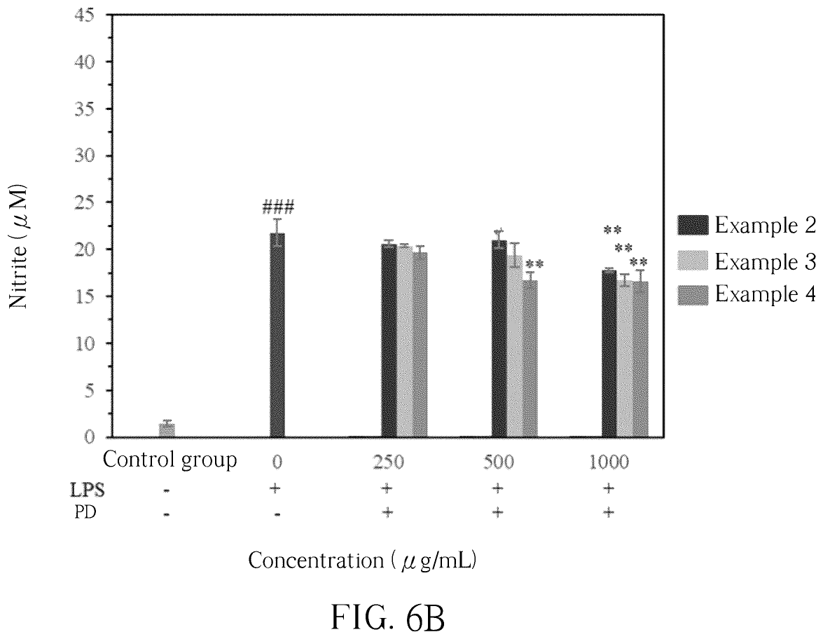

Each of RAW 264.7 cells and RAW 264.7 cells induced with LPS to generate NO were seeded at 5×10\hat{ }4 cells per well into 96-well plates and cultured for 24 hours to allow cell attachment. LPS and test samples at different concentrations were then added, followed by incubation at 37° C. for 24 hours. After incubation, the culture medium was replaced with medium containing 0.5 mg/mL MTT, and the plates were incubated at 37° C. for 4 hours. The resulting supernatant was collected and mixed with an equal volume of Griess reagent (1% sulphanilamide, 0.1% naphthylethylenediamine dihydrochloride, and 5% phosphoric acid). The absorbance at OD570 was measured using a multifunctional microplate spectrophotometer, and the cell viability of RAW 264.7 cells was calculated according to Equation (C), with the results shown in FIG. 6A. In addition, after the supernatant was removed, 200 L of DMSO was added to the remaining solution, and the absorbance at OD570 was measured. The cell viability of LPS-induced RAW 264.7 cells generating NO was calculated according to Equation (C), and the results are shown in FIG. 6B.

Referring to FIG. 6A, the polymer dots of Examples 2 to 4 did not induce NO production in any case, indicating that the animal-derived polymer dots of these examples did not elicit an inflammatory response in RAW 264.7 cells.

Referring to FIG. 6B, the polymer dots of Examples 2 to 4 did not induce NO production in any case, indicating that these samples did not trigger an inflammatory response in LPS-induced RAW 264.7 cells generating NO. In addition, when the sample concentrations of Examples 2 and 3 reached 1000 μg/mL, and when the sample concentration of Example 4 reached 500 μg/mL or higher, the samples were capable of scavenging the NO generated under LPS-induced inflammatory conditions. For example, compared with the positive control of 100 ng/mL LPS, treatment with 500 μg/mL of the animal-derived polymer dots of Example 4 resulted in an approximately 1.3-fold reduction in NO levels. These findings confirm that the animal-derived polymer dots of the present disclosure possess anti-inflammatory activity.

### 7. Free Radical Scavenging Capability

7.1 Free Radical: 1-Diphenyl-2-picrylhydrazyl (DPPH)

A solution having a concentration of 0.6 mg/mL was prepared by adding the animal-derived polymer dots of the Preparation Example and Examples 1 to 4 into double-distilled water. One milliliter of double-distilled water (blank control) or 1 mL of solutions at various concentrations was added to 2 mL of 1× phosphate-buffered saline (PBS) (pH 7.4), followed by the addition of 1 mL of 50 μM DPPH ethanol solution or 95% ethanol to maintain a fixed total volume, thereby obtaining the test samples. The mixtures were allowed to react in the dark for 30 minutes, after which 3 mL of each test sample was transferred into a quartz cuvette. The average absorbance at 517 nm was measured using a UV/Vis spectrophotometer, and the DPPH free radical scavenging rate was calculated according to Equation (D):

$$DPPH free radical scavenging rate ( \% ) = \{ 1 - [ ( {A}_{s} - {A}_{1} ) \div ( {A}_{c} - {A}_{0} ) ] \} \times 100 \% \qquad Equation ( D )$$

Aₛ: Absorbance of the Preparation Example or Example test sample with added DPPH ethanol solution
A₁: Absorbance of the Preparation Example or Example test sample with added 95% ethanol
A_{c}: Absorbance of double-distilled water with added DPPH ethanol solution
A₀: Absorbance of double-distilled water with added 95% ethanol

TABLE 5

| | DPPH Free Radical Scavenging Rate (%) of the |
|---|---|
| | Animal-Derived Polymer Dots (0.15 mg/mL) |
| Preparation Example | 7.4 ± 3.3 |
| Example 1 | 36.0 ± 4.9 |
| Example 2 | 40.7 ± 6.3 |
| Example 3 | 51.6 ± 7.3 |
| Example 4 | 41.6 ± 8.5 |

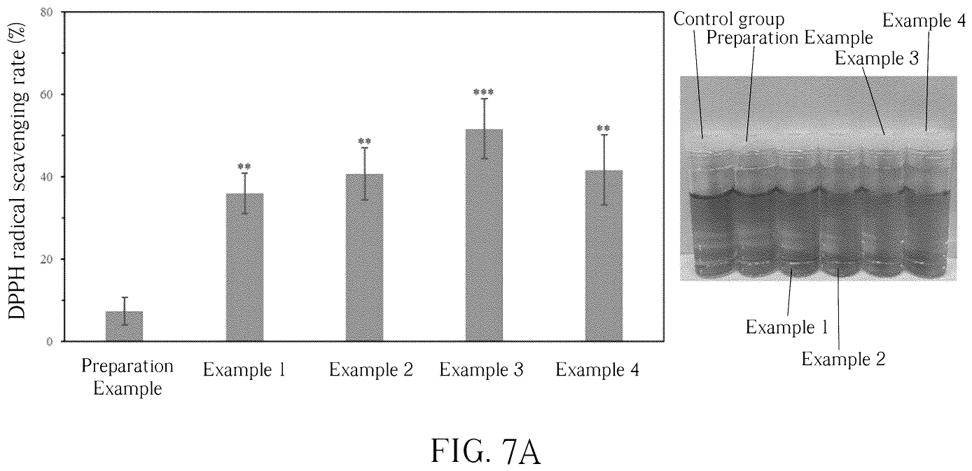

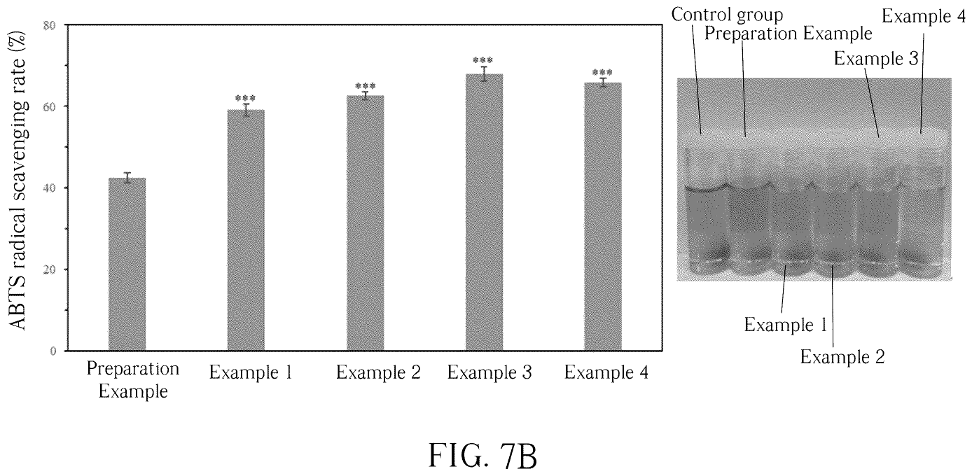

Referring to the left portion of FIG. 7A and Table 5, the polymer dots of the Preparation Example and Examples 1 to 4 each exhibited DPPH free radical scavenging activity. That is, the polymer dots of the Preparation Example and Examples 1 to 4 were capable of donating hydrogen ions to the DPPH free radicals, thereby enabling their scavenging. Consequently, the color of the solution changed from the blue-purple color characteristic of DPPH free radicals to a light yellow, as shown in the right portion of FIG. 7A.

Based on these results, the concentrations of the polymer dots of the Preparation Example and Example 3 required to achieve 50% scavenging activity, as well as the maximum scavenging activity, were further compared. The corresponding data are summarized in Table 6.

TABLE 6

| | Concentration at 50% Scavenging | Maximum Scavenging |
|---|---|---|
| | Activity (mg/mL) | Activity (%) |
| Preparation | 1.25 | 69.0 ± 10.0 |
| Example | | |
| Example 3 | 0.143 | 67.4 ± 6.6 |

As shown in Table 6, the animal-derived polymer dots of Example 3 achieved a scavenging rate comparable to that of the Preparation Example at a lower concentration, demonstrating superior DPPH free-radical scavenging capability.

7.2 Cationic Radical: 2,2′-Azino-bis(3-ethylbenzothiazoline-6-sulfonic acid) diammonium salt (ABTS)

Solutions of 0.1 mg/mL were prepared by adding the animal-derived polymer dots of the Preparation Example and Examples 1 to 4 to double-distilled water. A 14 mM ABTS solution was then added to a 4.9 mM potassium persulfate solution and incubated in the dark at 4° C. for 16 hours. The resulting ABTS cationic radical (ABTS·⁺) solution was diluted until its absorbance at 734 nm reached 0.7±0.02 L/(g cm). Subsequently, 1 mL of double-distilled water (blank group) or solutions of different concentrations was added to 2 mL of 1×PBS (pH 7.4), followed by the addition of 1 mL of the diluted ABTS·⁺ solution to obtain test samples. After incubation in the dark for 20 minutes, 3 mL of each test sample was transferred into a quartz cuvette, and the average absorbance at 734 nm was measured using a UV/Vis spectrophotometer. The ABTS cationic radical scavenging percentage was calculated according to Equation (E):

$$ABTS cationic radical scavenging ( \% ) = [ ( {A}_{c} - {A}_{s} ) \div {A}_{c} ] \times 100 \% \qquad Equation ( E )$$

Aₛ: Absorbance of the Preparation Example or Example test sample mixed with ABTS·⁺ solution.
A_{c}: Absorbance of double-distilled water mixed with ABTS·⁺ solution.

TABLE 7

| | ABTS Cationic Radical Scavenging Percentage (%) of the |
|---|---|
| | Animal-Derived Polymer Dots (0.025 mg/mL) |
| Preparation | 42.5 ± 1.2 |
| Example | |
| Example 1 | 59.1 ± 1.6 |
| Example 2 | 62.6 ± 0.9 |
| Example 3 | 68.0 ± 1.8 |
| Example 4 | 65.8 ± 1.1 |

Referring to the left portion of FIG. 7B and Table 7, the animal-derived polymer dots of the Preparation Example and Examples 1 to 4 each exhibit ABTS cationic radical scavenging activity. In other words, the polymer dots of the Preparation Example and Examples 1 to 4 were capable of reducing the ABTS cationic radical (ABTS·⁺) to ABTS, thereby enabling the ABTS cationic radical to be scavenged. As a result, the solution color shifted from the blue-green color of the ABTS cationic radical to a light blue-green or a colorless appearance, as shown in the right portion of FIG. 7B.

Based on these results, a further comparison was performed to determine the concentrations at which the animal-derived polymer dots of the Preparation Example and Example 3 achieved 50% scavenging activity, as well as their maximum scavenging activities. The data are summarized in Table 8 below.

TABLE 8

| | Concentration to Reach 50% | Maximum Scavenging |
|---|---|---|
| | Scavenging Activity (mg/mL) | Activity (%) |
| Preparation | 0.03 | 102.3 ± 2.7 |
| Example | | |
| Example 3 | 0.016 | 103.8 ± 0.8 |

As shown in Table 8, the animal-derived polymer dots of Example 3 achieved a scavenging rate comparable to that of the Preparation Example at a lower concentration, demonstrating superior ABTS radical scavenging ability.

These results confirm that the animal-derived polymer dots disclosed herein exhibit excellent DPPH and ABTS radical scavenging activity. Such activity enables the polymer dots to mitigate oxidative stress in cells and tissues caused by excessive free radicals in the body, thereby rendering them useful for preparing antioxidant compositions.

The foregoing description merely illustrates preferred embodiments of the present invention. Any equivalent modifications or alterations made according to the scope of the claims of the present invention shall fall within the scope of protection of the present invention.

What is claimed is:

1. An animal-derived polymer dot comprising:
a carbon dot represented by Formula (1): 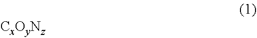
wherein C is carbon, O is oxygen, and N is nitrogen, and x, y, and z denote atomic percentages that satisfy 55≤x≤75, 15≤y≤35, 10≤z≤30, and x+y+z=100; and
wherein the carbon dot is derived from an animal-derived material.

2. The animal-derived polymer dot of claim 1, wherein x, y, and z satisfy 55≤x≤70, 20≤y≤35, 10≤z≤25, and x+y+z=100.

3. The animal-derived polymer dot of claim 1, wherein the carbon dot has a zeta potential of −30 mV to 0 mV.

4. The animal-derived polymer dot of claim 1, wherein the carbon dot has a particle size of 1 nm to 90 nm.

5. The animal-derived polymer dot of claim 1, wherein the carbon dot has an X-ray powder diffraction pattern comprising at least one characteristic peak with 20 values selected from the group consisting of 19.32°, 19.96°, 20.68°, 21.72°, 21.76°, 25.16°, 25.20°, 29.12°, 29.20°, 35.88°, 35.96°, 38.92°, and 39.04°±0.2°.

6. The animal-derived polymer dot of claim 1, wherein the carbon dot has:
an interplanar spacing of 3 Å to 5 Å corresponding to a graphite (002) plane; and/or
an interplanar spacing of 1 Å to 3 Å corresponding to a graphite (100) plane.

7. The animal-derived polymer dot of claim 1, wherein the carbon dot is formed by heating the animal-derived material by a hydrothermal method.

8. The animal-derived polymer dot of claim 1, wherein the animal is a deer.

9. The animal-derived polymer dot of claim 1, wherein the animal-derived material is deerhorn glue.

10. A method of preparing the animal-derived polymer dot of claim 1, comprising:
adding the animal-derived material into water to form an aqueous solution; and
heating the aqueous solution to obtain the animal-derived polymer dot.

11. The method of claim 10, further comprising hydrothermally treating the aqueous solution after the heating.

12. The method of claim 11, wherein the hydrothermal treatment comprises:
raising the temperature of the aqueous solution to a heating temperature of 150° C. to 250° C. at a pressure of 100 psi to 120 psi; and
maintaining the heating temperature for a heating time of 1 hour to 10 hours.

13. Use of the animal-derived polymer dot of claim 1 for preparing an anti-inflammatory composition, an antioxidant composition, or both.
